(19) United States
(12) Patent Application Publication
(10) Pub. No.: US 2011/0027916 A1
(43) Pub. Date: Feb. 3, 2011

(54) DETECTION APPARATUS FOR DETECTING PARTICLES

(75) Inventor: Jeroen Hans Nieuwenhuis, Waalre (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/936,411

(22) PCT Filed: Apr. 8, 2009
(86) PCT No.: PCT/IB2009/051481

§ 371 Date: Oct. 5, 2010

(30) Foreign Application Priority Data
Apr. 11, 2008 (EP) 08103493.6

(51) Int. Cl. G01N 33/553; G01N 33/48; B05D 5/00

(52) U.S. Cl. 436/526; 422/68.1; 422/69; 427/256

(57) ABSTRACT

The invention relates to a detection apparatus for detecting particles (25), wherein the detection apparatus comprises a detection surface (30) for detecting particles on the detection surface and wherein the detection surface (30) comprises protrusions (40) for limiting a movement of the particles (25). Preferentially, the protrusions (40) divide the detection surface into areas (41) surrounded by the protrusions such that a particle of the particles (25) located on the detection surface (30) is maintained within the respective area, if the particle moves laterally on the detection surface (30).

### FIELD OF THE INVENTION

The present invention relates to a detection apparatus, a detection method and a computer program for detecting particles. The invention relates further to a method for producing a detection apparatus for detecting particles.

### BACKGROUND OF THE INVENTION

As a detection apparatus for detecting particles biosensors are known. A biosensor detects particles by using, for example, a so-called sandwich assay. Magnetic beads are coated with a specific antibody that attaches to a target molecule present in a fluid like blood or saliva. When the magnetic beads, that are freely present in the fluid, have reacted with the available target molecules, the beads are attracted to a detection surface that has been coated with another antibody that can couple to the target molecule. The attraction force for attracting the magnetic beads with the attached target molecules to the detection surface is a magnetic force generated by a magnetic unit. After a sufficiently long reaction time the magnetic field is switched such that the magnetic beads, which are not bound to the detection surface, are pulled away from the detection surface, so that only the specifically bound magnetic beads with the target molecules, which are bound to the corresponding antibodies, remain attached to the detection surface. The magnetic beads on the detection surface can be detected magnetically or optically.

This biosensor has the drawback that the magnetic beads are gathered at certain locations on the detection surface, which are defined by an inhomogeneity of the magnetic field generated by the magnetic unit. Thus, the probability that a magnetic bead is bound on the detection surface is reduced at the locations, at which the magnetic beads are gathered due to the inhomogeneous magnetic field, in comparison to other locations on the detection surface. This reduces the sensitivity of the detection apparatus in these locations and, thus, the overall sensitivity of the detection apparatus.

### SUMMARY OF THE INVENTION

It is an object of the present invention to provide a detection apparatus, a detection method and a computer program for detecting particles, wherein the sensitivity of detecting the particles is increased. It is a further object of the present invention to provide a corresponding method for producing a detection apparatus for detecting particles.

In a first aspect of the present invention a detection apparatus for detecting particles is presented, wherein the detection apparatus comprises a detection surface for detecting particles on the detection surface and wherein the detection surface comprises protrusions for limiting a movement of the particles.

The invention is based on the idea that the inhomogeneous distribution of magnetic particles on the detection surface is caused by a movement of the particles along the detection surface. Since the forces, which move particles along the detection surface are generally not homogeneous, the movement along the detection surface yields an inhomogeneous distribution of the particles on the detection surface. Because of this inhomogeneous distribution of particles on the detection surface, in the prior art, the sensitivity of the detection at locations on the detection surface, at which the particles are gathered, is reduced in comparison to other locations, thereby reducing the overall sensitivity of the detection apparatus. This effect of the inhomogeneous distribution of the particles can be diminished by providing protrusions on the detection surface, which limit the movement of the particles. Thus, particles, which have to be detected on the detection surface, cannot freely move along the detection surface and gather at certain locations, which are defined by inhomogeneous forces, which move the particles. Thus, by providing the protrusions for limiting a movement of the particles the distribution of the particles on the detection surface can be more homogeneous, thereby increasing the sensitivity of the detection apparatus for detecting particles.

The protrusions are preferentially adapted for limiting a lateral movement of the particles along the detection surface. Since the inhomogeneous distribution of particles in the state of the art is supposed to be caused mainly by a lateral movement of the particles along the detection surface, the adaptation of the protrusions such that they limit the lateral movement of the particles will further increase the degree of homogeneity of the distribution of particles on the detection surface.

The detection apparatus is preferentially a biosensor, and the particles are preferentially magnetic particles.

It is further preferred that the protrusions divide the detection surface into areas surrounded by the protrusions such that a particle of the particles located on the detection surface is maintained within the respective area, if the particle moves laterally on the detection surface. Since the particles cannot move freely in any direction parallel to the detection surface and since a main reason for an inhomogeneous distribution of the particles is supposed to be a movement along and parallel to the detection surface, the surrounded areas further increase the degree of homogeneity of the distribution of particles on the detection surface and, thus, the sensitivity of the detection apparatus.

It is further preferred that the detection surface comprises binding sites for binding the particles. This further reduces a movement of the particles and, thus, further increases the degree of homogeneity of the distribution of particles on the detection surface. Furthermore, since the binding sites bind the particles on the detection surface, they are hold on the detection surface for detecting these particles, which further improves the sensitivity of the detection apparatus.

In a preferred embodiment, the detection surface, comprises different kinds of binding sites comprising different binding properties. This allows to differentiate between different particles or the same particles with different attaching elements for attaching the particle to a target element, wherein the binding property of a binding site can be specific for the target element, to which the attaching element of the particle has been attached.

The detection apparatus comprises preferentially a magnetic unit, wherein the particles are magnetic particles and wherein the magnetic unit is adapted for generating a magnetic field for forcing the particles onto the detection surface. Since the particles are forced onto the detection surface by the magnetic field, more particles are present on the detection surface, wherein the sensitivity of the detection apparatus is further increased.

In a preferred embodiment, the detection apparatus comprises a magnetic unit for moving the particles laterally on the detection surface. Thus, the movement is limited by the protrusions, but between the protrusions the particles are moved laterally along the detection surface, thereby increasing the probability of binding a particle on a binding site, which might be present between the protrusions and which can hold the particle at the binding site, wherein the sensitivity of the detection apparatus is further increased.

The magnetic unit for moving the particles laterally on the detection surface and the magnetic unit for generating a magnetic field for forcing the particles onto the detection surface can be the same magnetic unit, or the magnetic unit for moving the particles laterally on the detection surface and the magnetic unit for generating a magnetic field for forcing the particles onto the detection surface can be separate magnetic units.

It is further preferred that the detection apparatus comprises a magnetic unit for generating a magnetic field for forcing the particles, which are not bound to the detection surface, away from the detection surface. Since particles, which are not bound to the detection surface, are forced away from the detection surface, only particles are detected, which are bound to the detection surface, i.e. only particles with certain attaching properties with respect to the detection surface are detected. This allows to detect a certain type of particles, i.e. which are bindable to the detection surface at the binding sites. This forcing of the particles away from the detection surface can be regarded as washing step.

It is further preferred that the detection apparatus comprises a particle providing unit for providing the particles, wherein the particle providing unit is adapted for providing particles, which comprise an attaching element for attaching the particle to a target element, wherein the detection surface comprises binding sites for binding the particles and wherein the binding sites are adapted such that a particle of the particles is bindable to a binding site, if the particle is attached to the target element. In particular, the target element is bindable to a binding site and since the target element is attached to the particle via the attaching element, the particle is bindable to the binding site via the target element. This allows to detect the target element, for example, to determine the concentration of the target element in a fluid, by detecting the particle.

In a preferred embodiment, the detection apparatus comprises a detection cavity, in which a fluid comprising target elements and the particles provided by the particle providing unit are introducable for allowing the attaching elements of the particles to attach to the target elements of the fluid, wherein the detection apparatus is adapted for exposing the fluid comprising the target elements attached to the particles to the detection surface comprising the binding sites. In particular, an inner surface of the detection cavity is the detection surface. This allows to detect particles and elements attached to the particles within a fluid like blood or saliva.

It is further preferred that the protrusions define areas, wherein in at least one area a binding site is located, which differs from a binding site located in another at least one area with respect to binding properties, wherein the detection apparatus comprises different particle providing units for providing particles with different kinds of attaching elements, wherein the different kinds of attaching elements correspond to the different kinds of binding sites such that a particle with an attaching element binds mainly to the corresponding binding site. In particular, different kinds of attaching elements are attachable to different target elements and the different target elements, to which the particles are attached, are bindable to corresponding binding sites, i.e. a sandwich structure consisting of the particle, the target element and the binding site is used for binding a particle with an attaching element mainly to the corresponding binding site. This allows to detect different target elements, which are attached to the particles via the different attaching elements and which are bound at the different kinds of binding sites on the detection surface.

In a preferred embodiment, in a same area of the areas on the detection surface a particle providing unit is located, which provides particles with a kind of attaching elements, which corresponds to a binding site in the same area. It is also preferred that the detection apparatus comprises a particle providing surface, on which the particle providing units are located, wherein the particle providing surface is located opposite to the detection surface and wherein the particle providing units are arranged opposite to the corresponding binding sites. These arrangements of particle providing units and corresponding binding sites reduces cross-talk, i.e. the probability of binding a particle comprising an attaching element to a binding site, which does not correspond to this attaching element. Thus, the probability of binding a particle comprising an attaching element, which has been attached to a certain kind of target element, to a binding site, which does not correspond to the attaching element and, thus, to the target element attached to the attaching element, is reduced.

It is further preferred the detection apparatus comprises a particle detection unit for detecting the particles on the detection surface. The particles are preferentially magnetic particles and the magnetic particles are preferentially detected magnetically or optically by the particle detection unit.

It should be understood that the detection apparatus comprises the particle detection unit in an embodiment only and that the detection apparatus can be an apparatus without the particle detection unit, i.e. the detection apparatus can be an apparatus for detecting particles, which comprises a detection surface for detecting particles on the detection surface, wherein the detection surface comprises protrusions for limiting a movement of the particles and wherein a particle detection unit can be a separate part, which does not belong to the detection apparatus. For example, the detection apparatus comprising the detection surface with the protrusions can be disposable, for example, can be a disposable cartridge comprising the detection surface, which can be inserted in a particle detection unit for detecting particles on the detection surface.

In a further aspect of the present invention a detection method for detecting particles is presented, wherein the detection method comprises the steps of detecting particles on a detection surface comprising protrusions for limiting a movement of the particles.

In a further aspect of the present invention a method for producing a detection apparatus for detecting particles is presented, wherein the method comprises following steps:
providing a detection surface material for producing a detection surface for detecting particles on the detection surface,
forming a detection surface from the provided detection surface material such that the detection surface comprises protrusions for limiting a movement of the particles.

In a further aspect of the present invention a computer program for detecting particles is presented, wherein the computer program comprises program code means for causing a detection apparatus as defined in claim 1 to carry out the steps of the detection method as defined in claim 13, when the computer program is run on a computer controlling the detection apparatus.

It shall be understood that the detection apparatus of claim 1, the detection method of claim 13, the method for producing a detection apparatus of claim 14 and the computer program of claim 15 have similar and/or identical preferred embodiments as defined in the dependent claims.

It shall be understood that a preferred embodiment of the invention can also be any combination of the dependent claims with the respective independent claim.

### BRIEF DESCRIPTION OF THE DRAWINGS

These and other aspects of the invention will be apparent from and elucidated with reference to the embodiments described hereinafter. In the following drawings:

FIG. 1 shows schematically and exemplarily an embodiment of a detection apparatus for detecting particles,

FIG. 2 shows schematically and exemplarily a sectional view of a cartridge of the embodiment of the detection apparatus for detecting particles,

FIG. 3 shows schematically and exemplarily a double-sided tape of the cartridge,

FIG. 4 shows schematically and exemplarily a top view on the cartridge,

FIG. 5 shows schematically and exemplarily a top view on separate elements of the cartridge,

FIG. 6 shows schematically and exemplarily a sectional view of a cartridge of a detection apparatus for detecting particles,

FIG. 7 shows schematically and exemplarily a sectional view of a part of a detection cavity comprising a detection surface,

FIG. 8 shows schematically and exemplarily a sectional view of a part of a detection cavity comprising a detection surface,

FIG. 9 shows schematically and exemplarily a top view on the detection surface shown in FIG. 8,

FIG. 10 shows schematically and exemplarily a sectional view through a detection cavity comprising the detection surface shown in FIGS. 8 and 9,

FIG. 11 shows schematically and exemplarily a sectional of a part of a detection cavity comprising a detection surface,

FIG. 12 shows schematically and exemplarily a sectional view through a cavity comprising the detection surface shown in FIG. 11,

FIG. 13 shows schematically and exemplarily a top view on the detection surface shown in FIGS. 11 and 12,

FIG. 14 shows a flow chart illustrating an embodiment of a detection method for detecting particles, and

FIG. 15 shows a flow chart illustrating a method for producing a detection apparatus for detecting particles.

### DETAILED DESCRIPTION OF EMBODIMENTS

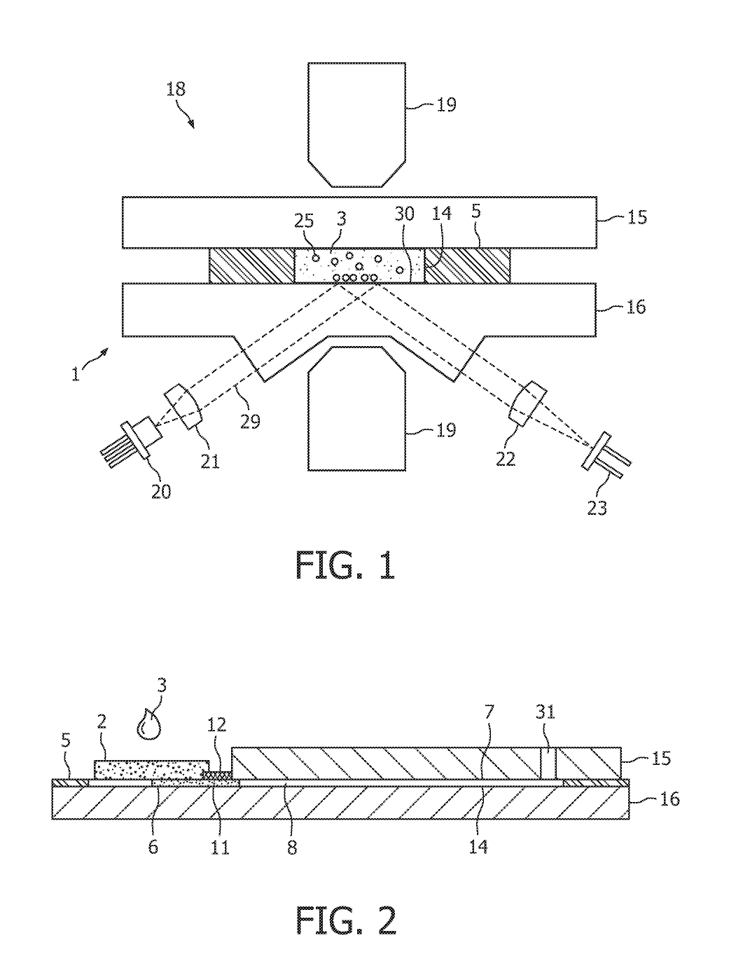

FIG. 1 shows schematically and exemplarily a detection apparatus 1 for detecting particles 25. The detection apparatus 1 comprises a detection surface 30 for detecting particles on the detection surface 30. The detection apparatus 1 further comprises a detection cavity 14, in which a fluid 3, in which the particles 25 are present, can be introduced. The fluid 3 is preferentially blood or saliva and the particles 25 are, in this embodiment, magnetic particles.

The detection cavity 14 is, in this embodiment, formed by a double-sided tape 5, i.e. which has adhesive properties at two opposing sides, a base element 16 and a cover element 15, wherein the double-sided tape 5 is located between the cover element 15 and the base element 16. The double-sided tape 5 comprises a gap, in which the detection cavity 14 is formed. The base element 16, the cover element 15 and the double-sided tape 5, which is adhesive at the two sides, which face the base element 16 and the cover element 15, form a cartridge comprising the detection surface 30.

The cartridge, which is a part of the detection apparatus, is schematically and exemplarily shown in FIG. 2. The cartridge comprises a filter element 2 for filtering the fluid 3 and the adhesive double-sided tape 5, in which a capillary structure is generated for generating capillary forces. The double-sided tape 5 is attached to the filter element 2 by using the adhesive properties of the double-sided tape.

The cartridge further comprises a filtering location 6, at which the filter element is located, and a detection location 7, at which the detection cavity 14 is located, wherein the double-sided tape 5 is formed such that the filtered fluid 3 is guided from the filtering location 6 to the detection location 7.

The double-sided tape 5 comprises a connecting channel 8, which connects the filtering location 6 with the detection location 7, wherein the double-sided tape 5 further comprises guiding channels 9 located at the filtering location 6 and wherein the guiding channels 9 extend from an end 10 of the connecting channel 8. In this embodiment, the guiding channels 9 extend radially from the end 10 of the connecting channel 8. Furthermore, in this embodiment, the guiding channels 9 have a smaller width than the connecting channel 8. In this embodiment, the connecting channel 8 has two widths, a smaller width between the end 10 and the detection location 7 and a larger width at the detection location 7 for forming the detection cavity 14.

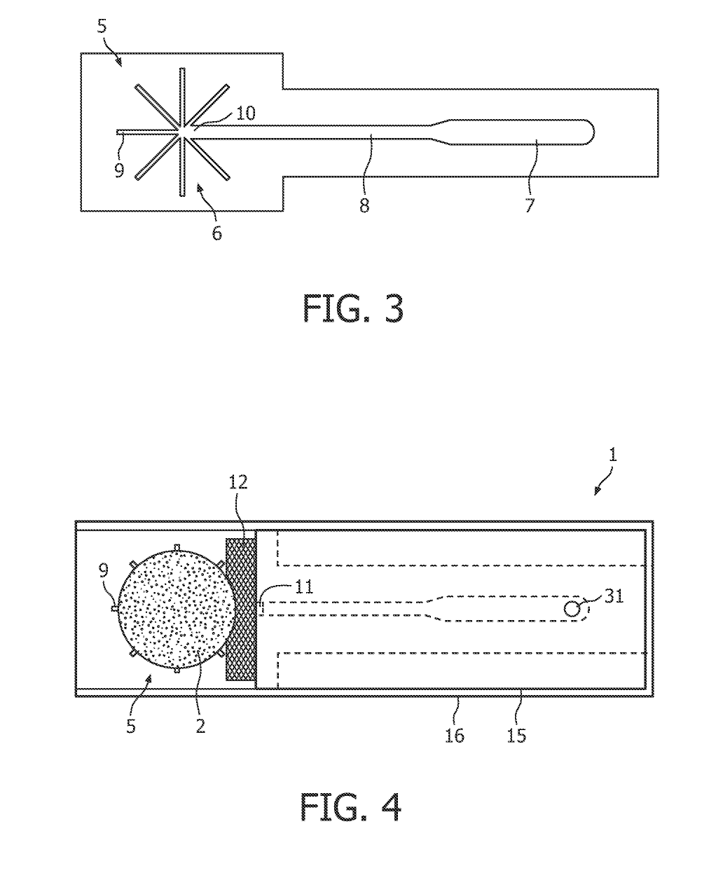

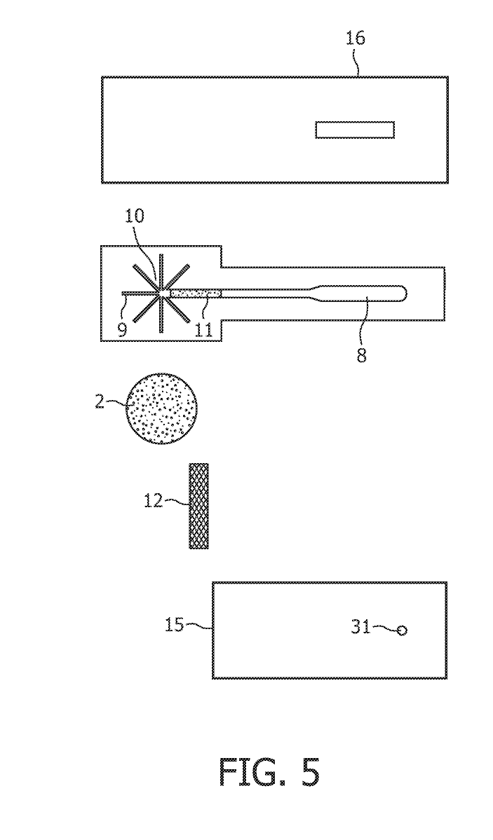

The guiding channels 9 extend to a radius outside the filter element 2. This can be seen in FIG. 4 showing a top view on the cartridge, which is shown in a sectional view in FIG. 2. A top view on the double-sided tape 5 is shown in FIG. 3. FIG. 5 shows separate parts of the cartridge schematically and exemplarily.

FIG. 5 shows a transferring element 11 for transferring the fluid 3 in the direction of the detection location 7. The transferring element 11 is preferably a cellulose strip located in the connecting channel 8 adjacent to the end 10 of the connecting channel 8. In other embodiments, the transferring element 11, in particular the cellulose strip, could be located in at least one of the guiding channels 9 or in both, at least one of the guiding channels 9 and in the connecting channel 8. In other embodiments, it may consist of a micro-fluidic structure, in particular micro extrusions or grooves, that increase the capillary forces and effectively guides a fluid into the connecting fluidic channel towards the detection cavity 14.

The cartridge preferentially further comprises a bypass preventing unit 12 for preventing the fluid 3 from bypassing the filter element 2 and flowing directly into the connecting channel 8. The bypass preventing unit 12 is a strip, which is preferentially made of a material, through which the fluid cannot pass and which is located between the connecting channel 8 and the filter element 2 to exclude the possibility that a part of the fluid sample bypasses the filter element 2. In another embodiment, the filter element is extended in the direction of the connecting channel such that a direct bypass in the connecting channel is prevented.

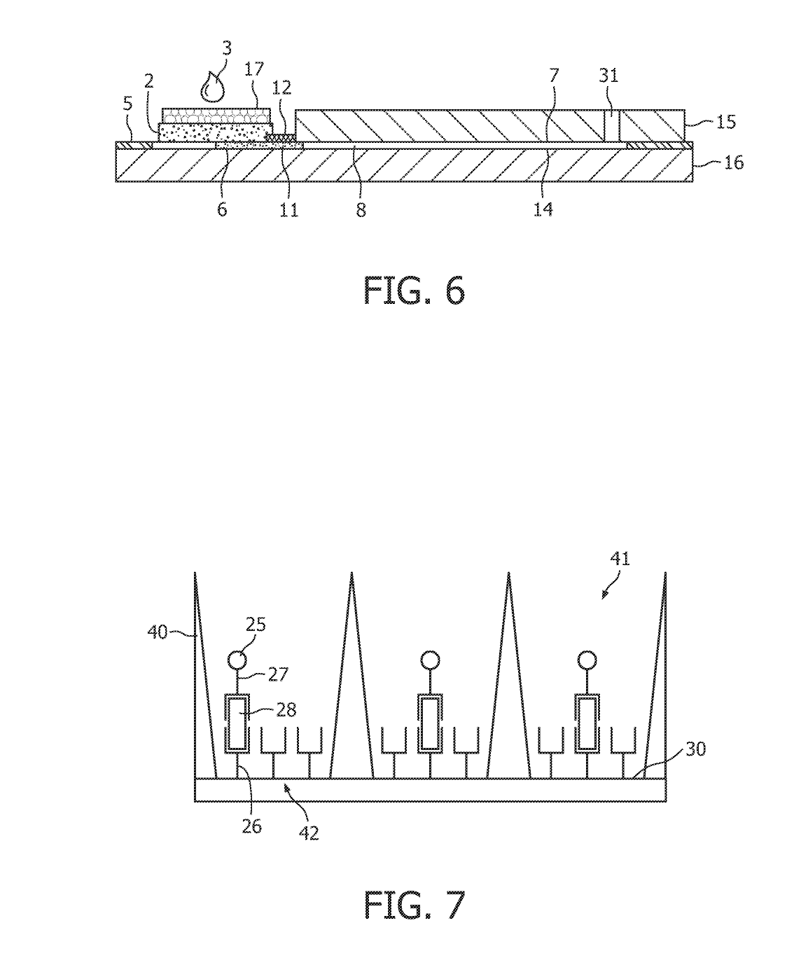

The base element 16 and the cover element 15 are preferentially plastic elements, which are injection moulded and preferentially transparent for visible light. The cartridge optionally comprises a deposition medium 17, which is arranged on the filter element 2, for deposition of the fluid 3 on the filter element 2. An embodiment with a deposition medium 17 is schematically and exemplarily shown in FIG. 6.

The cover element 15 comprises a vent 31 for allowing a gas to leave the cartridge.

In this embodiment, the filter element 2 is a blood-separation filter and the cartridge is preferentially disposable. The blood-separation filter is preferentially a cross-flow blood-separation filter. The detection apparatus comprising the cartridge is preferentially used in point-of-care diagnostics.

The cartridge and, thus, the detection apparatus are preferentially adapted for detecting low concentration biomarkers in a sample of whole blood, in particular in a finger prick sample of, for example, 25 μL. The detection cavity 14 preferentially comprises an immuno assay.

The filter element 2 is preferentially adapted for extracting blood plasma from the whole blood to guarantee optimal functioning of the immuno assay. Furthermore, the cartridge is preferentially adapted such that only capillary forces are the driving forces for fluid transport, in order to keep the cartridge simple, robust and inexpensive. Furthermore, the capillary structure in the double-sided tape, which contains preferentially a cellulose strip, is preferentially fine enough to generate enough driving force to pull the plasma fraction of the whole blood sample through the filter element. The filter element is preferentially adapted as a cross-flow filter, wherein the cross-flow filter is large enough for filtering without clogging and for inducing a moderate pressure drop. The attached and preferentially underlying capillary structure comprises preferentially a volume which is small enough for generating the necessary capillary force by means of narrow channels.

The filter element 2 is preferentially an asymmetric membrane filter, for example, a Pall filter. The filter element is preferentially a cross-flow filter having a large pore diameter at the side, on which the fluid is to be placed, of about 100 μm and a small pore diameter of about 1 μm at the opposite side of the filter element, so that the cells of the blood are obstructed by size exclusion.

Referring again to FIG. 1, the detection apparatus comprises a particle detection unit 20 . . . 23 for detecting the particles 25 on or near the detection surface 30. The particle detection unit comprises a light source 20, which is, for example, a laser device or a LED, for generating a light beam 29, which illuminates the detection surface 30. The particle detection unit further comprises a detector 23 for detecting the light reflected from the detection surface. In this embodiment, the particle detection unit further comprises optical elements 21 and 22, which are arranged in the light beam 29 for generating parallel light or focussing the light beam 29, respectively. The optical elements 21, 22 are preferentially lenses.

The particle detection unit shown in FIG. 1 is adapted for detecting changes at the detection surface 30 using the so called FTIR method (Frustrated Total Internal Reflection). The beam of light is reflected on an interface between a medium with a higher refractive index, for example, the base element 16, and a medium with a lower refractive index, for example, the fluid. There is a certain critical angle of incidence above which there is a situation of total internal reflection (TIR). The present detection configuration (regarding refractive indices and angle of incident) is such that there is a total internal reflection of the incoming beam. Although the light is totally reflected in such a situation, there is still a penetration of the light in a very thin layer of the medium with the lower refractive index. This is the so called evanescent light, the intensity of which decays exponentially in the low refractive index medium with a characteristic penetration depth in the order of the wavelength of the light. Thus, in practise the penetration depth is preferentially less than 0.5 micrometer. If particles, in particular, magnetic particles, are bound to the detection surface, the optical properties of this very thin first fluid layer of preferentially about 0.5 micrometer are changed leading to a reduction of the reflected light beam. This is caused by absorption and scattering of the evanescent light (FTIR). As a result the signal of the detector 23 changes. This change of the signal of the detector 23 can be related to the amount of particles on the detection surface, for example, by calibration measurements. Thus, by detecting the change of the signal of the detector 23, the amount of particles on the detection surface can be determined.

The detection surface 30 of the detection cavity 14 comprises protrusions 40, which are schematically and exemplarily shown in FIG. 7.

FIG. 7 shows schematically and exemplarily the detection surface 30 of the detection cavity 14. The detection surface 30 comprises protrusions 40, which divide the detection surface 30 into areas 41. The protrusions 40 limit a movement of the particles 25 along the detection surface 30, i.e. they limit the lateral movement of the particles 25. The protrusions 40 divide the detection surface 30 into areas 41 surrounded by the protrusions 40 such that a particle 25 located on the detection surface 30 is substantially maintained within the respective area, if the particle moves laterally on the detection surface 30. The protrusions 40 surrounding an area 41 form preferentially a rectangular area. The protrusions 40 reduce the probability of a concentration of the particles 25 on certain locations on the detection surface 30.

The detection surface 30 comprises binding sites 42 for binding the particles 25. In particular, the binding sites 42 comprise binding elements 26, which are adapted for binding a target element 28, which can be attached to an attaching element 27 comprising the particle 25. The target element 28 is, for example, a target molecule within a fluid and the binding element 26 and the attaching element 27 are preferentially antibodies with respect to the target molecule. An assay using such a sandwich structure can be denoted as sandwich assay.

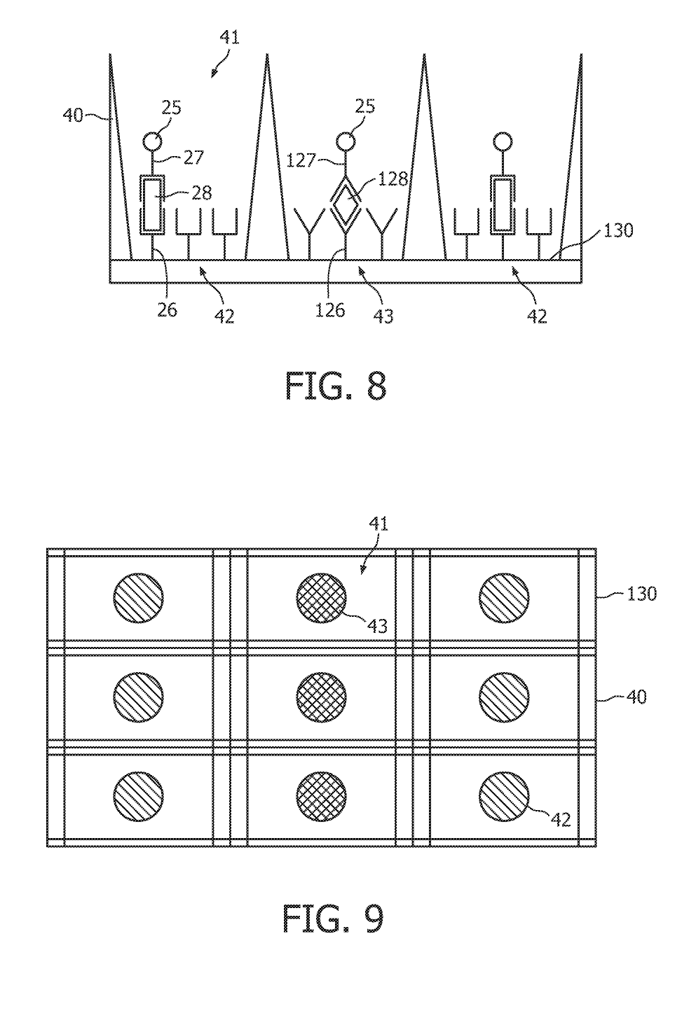

FIG. 8 shows schematically and exemplarily another embodiment of a detection surface 130, which can be present within the detection cavity 14. Also in this embodiment, the detection surface 130 comprises protrusions 40, which divides a detection surface 130 into areas 41 surrounded by the protrusions 40 such that a particle 25 located on the detection surface 130 is substantially maintained within the respective area 41, if the particle moves laterally on the detection surface 130.

The detection surface 130 comprises two different kinds 42, 43 of binding sites comprising different binding properties. The binding site 42 comprises binding elements 26, which bind specifically to the target elements 28, while the binding site 43 comprises binding elements 126, which bind specifically to target elements 128.

In the embodiment shown in FIG. 7, the detection apparatus comprises a particle providing unit for providing the particles 25, which comprise the attaching element 27 for attaching the respective particle 25 to a target element 28. The particle providing unit can, for example, be located on an inner surface of the detection cavity 14, in particular, on an inner surface opposite to the detection surface or on the detection surface itself. The particle providing unit can comprise a group of the particles 25 comprising the attaching elements 27, which are loosely attached at a location on the inner surface of the detection cavity 14 such that they can leave the inner surface and immerse into a fluid comprising target elements, if the inner surface is exposed to the fluid.

In the embodiment shown in FIG. 8, two particle providing units of the detection apparatus provide particles 25 with two different kinds of attaching elements 27, 127. The attaching element 27 is adapted for attaching to the target element 28 and the attaching element 127 is adapted for attaching the target element 128. This allows to bind the particles 25 via the target element 28 at the binding sites 42 and to bind the particles 25 via the target elements 128 at the binding sites 43. Thus, by determining the amount of particles 25 at the binding sites 42, the amount of target elements 28 bound on the detection surface 130, and by determining the amount of particles 25 at the binding sites 43, the amount of target elements 128 bound to the detection surface 130 can be determined.

FIG. 9 shows schematically and exemplarily a top view on the detection surface 130 shown in FIG. 8. The protrusions 40 divide the detection surface 130 into several rectangular areas 41, wherein in a rectangular area 41 a binding site 43 or 42 is located. In this embodiment, the binding sites are circularly shaped and in the same column either the binding sites 42 or the binding sites 43 are located only.

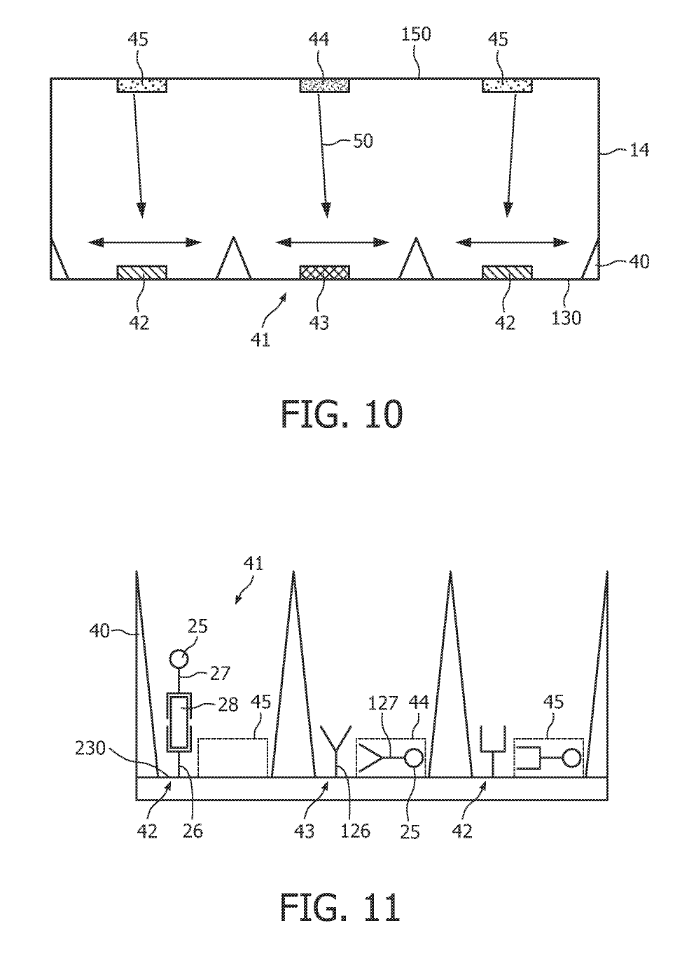

FIG. 10 shows schematically and exemplarily a sectional view through the detection cavity 14 in an embodiment comprising the detection surface 130. In this embodiment, particle providing units 44, 45 are located on a particle providing surface 150, which is arranged opposite to the detection surface 130, in particular, the particle providing units 44, 45 are two different particle providing units, which provide particles 25 comprising the attaching elements 127 and particles 25 comprising the attaching elements 27, respectively. Thus, two different kinds of particle providing units are located on the particle providing surface 150, wherein the particle providing unit 44, which provides particles 25 comprising the attaching elements 127, is arranged opposite to the corresponding binding site 43 and wherein the particle providing unit 45 providing the particles 25, which comprise the attaching elements 27, is located opposite to the corresponding binding site 42.

If a fluid is introduced into the detection cavity 14, the particles 25 comprising the attaching elements 27 and 127, are in contact with the fluid and immerse into the fluid. The attaching elements 27, 127 attach to the corresponding target elements 28, 128 within the fluid and the particles 25 comprising the attaching elements 27, 127 are forced to move to the corresponding binding sites 42, 43 by a magnetic field generated by the magnetic unit 19. A movement of a particle 25 comprising an attaching element from a particle providing unit 44 to a corresponding binding site 43 is schematically and exemplarily indicated in FIG. 10 by the arrow 50. Since the particle providing units are arranged opposite to the corresponding binding sites, since the magnetic field moves the particles 25 comprising the attaching elements substantially normal to the detection surface 130, since each area 41 surrounded by the protrusions 40 comprises preferentially only a single kind of binding site and since the protrusions 40 prevent that particles 25 comprising the attaching elements 27, 127 can leave area 41 by lateral movement on the detection surface 130, the probability of a cross talk can be reduced, i.e. the probability can be reduced that a particle 25 comprising an attaching element is detected at a binding site, which does not correspond to the respective attaching element, i.e. which does not correspond to the respective particle providing unit, which has provided the particle comprising the attaching element.

FIG. 11 shows schematically and exemplarily another embodiment of a detection surface 230, which can be an inner surface of the detection cavity 14. Also the detection surface 230 comprises protrusions 40, which divide the detection surface 230 into rectangular areas 41. In this embodiment, on the detection surface 230 two kinds 42, 43 of binding sites are located. The binding site 42 comprises a binding element 26 and the binding site 43 comprises a binding element 126. In this embodiment, within a single area 42 only one kind of a binding site, 42 or 43, and a corresponding particle providing unit, 45 or 44, is located on the detection surface. The particle providing units 44, 45 are schematically indicated by the broken line in FIG. 11. In the left area 41 shown in FIG. 11 the particle 25 comprising the attaching element 27 has been provided by the particle providing unit 45, the attaching element 27 has attached to a target element 28 and the combination of the target element 28, the attachment element 27 and the particle 25 is bound to the detection surface 230 via the binding element 26. In FIG. 11 in the middle area and in the area shown on the right hand side, particles comprising an attachment element are still present in the particle providing unit and can be provided by the particle providing unit.

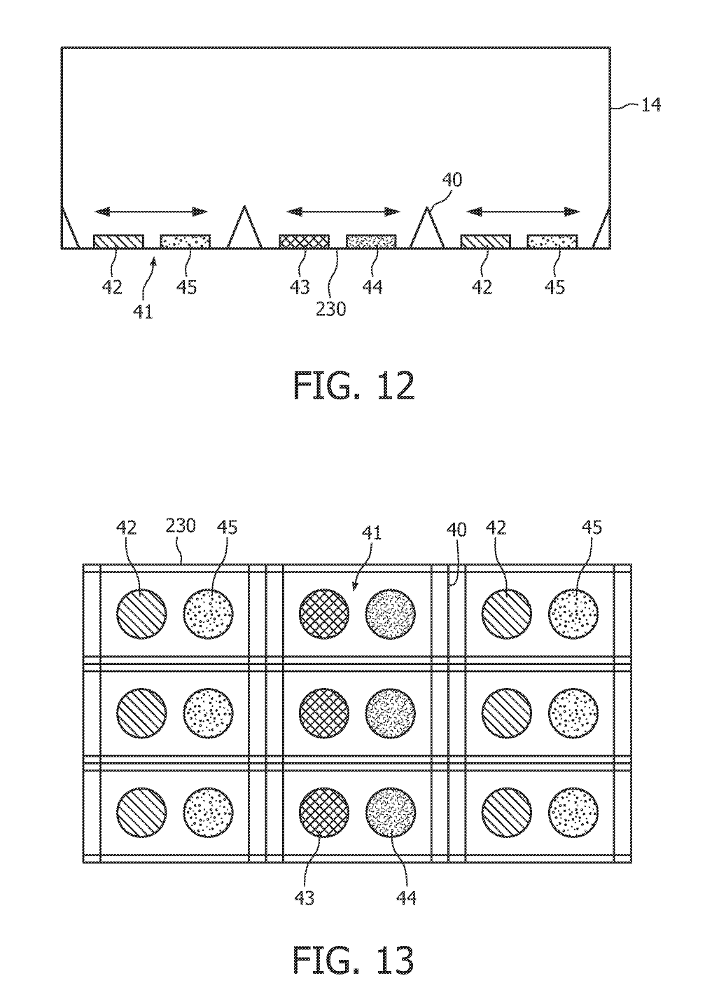

FIG. 12 shows schematically and exemplarily a sectional view of the cavity 14 comprising the detection surface 230 with the protrusions 40 defining areas 41, wherein in each area 41 only one kind of a binding site, 42 or 43, and a corresponding particle providing unit, 44 or 45, is located. FIG. 13 shows schematically and exemplarily a top view on the detection surface 230 comprising the protrusions 40 defining areas 41, wherein in each area 41 only one kind of a binding site, 42 or 43, and a corresponding particle providing unit, 44 or 45, is located.

If in this embodiment a fluid comprising target elements 28, 128 is introduced into the cavity 14, the particles 25 comprising the attaching elements 27, 127 provided by the particle providing units 44, 45 immerse into the fluid and attach to the corresponding target elements 28, 128. Furthermore, the target elements 28, 128 bind to the corresponding binding elements 26, 126, thereby binding the particles 25 comprising the attaching elements 27, 127, which have been attached to the corresponding target elements 28, 128, which are bound to the corresponding binding elements 26, 126 on the detection surface 230. The particles 25 on the detection surface can be detected by the detection apparatus 20 . . . 23. Since the magnetic field of the magnetic unit 19 is adapted such that the particles 25 move only laterally on the detection surface 230, they cannot leave the area 41 because of the protrusions 40 surrounding the respective areas 41 and can thus only be bound to the corresponding binding site located within the same area 41. This reduces the probability of cross talk, i.e. the probability that a particle 25 comprising an attaching element is bound to a binding site, which does not correspond to the attaching element, i.e. which does not correspond to the particle providing unit, which provides the particles comprising the respective attaching element.

The magnetic unit is preferentially adapted such that during an attaching and binding phase the particles, which are not located on the detection surface, are forced substantially normal to the detection surface onto the detection surface and such that the particles, which are located on the detection surface and which are not bound to the detection surface, are moved laterally on the detection surface. In a washing phase, which follows the attaching and binding phase, the magnetic unit generates a magnetic field, which pulls the particles, which are not bound to the detection surface, away from the detection surface such that during a detection phase, which preferentially follows the washing phase only particles are detected on the detection surface, which are bound at the binding sites on the detection surface.

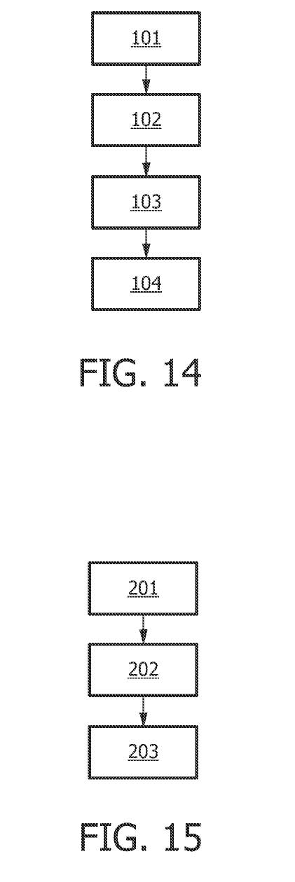

In the following an embodiment of a detection method for detecting particles will be exemplarily described with reference to a flow chart shown in FIG. 14.

In step 101, a fluid is introduced into the cavity 14 and the particle providing units provide particles comprising attaching elements, which immerse into the fluid. The attaching elements attach to the target elements and in step 102 the magnetic unit 19 forces the particles 25 onto the detection surface. In addition, the magnetic unit moves the particles along the detection surface within the respective area, in which the respective particle is located. The particles comprising the attaching elements, which have been attached to a target element, are bound by the binding elements at the corresponding binding sites.

In step 103, particles, which are not bound on the detection surface, are forced away from the detection surface by the magnetic field of the magnetic unit.

In step 104, the particle detection unit detects the particles bound on the detection surface. The detected amount of particles on the detection surface can directly be related to the amount of target elements, which are sandwiched by the binding elements and the attaching elements. Thus, by determining the amount of particles, in particular, the amount of magnetic particles, on the detection surface, the amount of target elements on the detection surface and, thus, in the fluid can be determined.

In the following, an embodiment of a method for producing a detection apparatus for detecting particles will exemplarily be described with reference to a flow chart shown in FIG. 15.

In step 201, a detection surface material is provided for producing a detection surface for detecting particles on the detection surface. In step 202, a detection surface is formed from the provided detection surface material such that the detection surface comprises protrusions for limiting a movement of the particles, which have to be detected. In step 203, a particle detection unit is provided for detecting particles, which are present on the detection surface.

In an embodiment, the detection apparatus is a biosensor based on the magnetic detection of super-paramagnetic labels on the detection surface, which can also be called beads and which are preferentially the particles. Instead of magnetic detection the magnetic particles can also be detected optically, for example, by the above described particle detection unit, which uses the FTIR technique.

The detection apparatus is preferentially adapted to detect specific target molecules, like drugs or cardiac markers, in a fluid, like saliva or blood, by determining the amount of particles, in particular, magnetic particles, bound at binding sites on the detection surface. The binding elements are preferentially antibodies and/or drug molecules, which specifically bind to the target molecules, to which an attaching element with a magnetic particle has been attached.

In particular, the particles are magnetic beads and the attaching elements are primary antibodies on the magnetic beads, and at the binding sides secondary antibodies are present. The primary and secondary antibodies bind to different parts of the analyte (target element), i.e. the primary and secondary antibodies are preferentially primary and secondary anti-PTH/anti-troponin antibodies, respectively.

If in other embodiments an inhibition assay is used instead or in addition to a sandwich assay, the particles comprising attaching elements are, for example, opiate magnetic particles, amphetamine magnetic particles, cocaine magnetic particles or methamphetamine magnetic particles and the corresponding binding sites are, for example, opiate conjugated with bovine serum albumin, amphetamine conjugated with bovine serum albumin, cocaine conjugated with bovine serum albumin and methamphetamine conjugated with bovine serum albumin.

Although in the embodiment described above with reference to FIGS. 11 to 13 corresponding binding sites and particle providing units are located already within the same area on the detection surface, in this and also in other embodiments, the magnetic unit does not only generate lateral magnetic forces, but has preferentially also a force component that is directed towards the detection surface. This normal or perpendicular force component ensures that the particles will stay close to the detection surface. The lateral component of the magnetic forces will move the particles laterally over the detection surface until they hit a protrusion, which blocks the lateral movement. The lateral magnetic force is therefore preferentially altered such that the blocked particle moves in another direction, until they run into other protrusions. By an appropriate sequence of lateral movements the particles have interaction with their complementary binding sites a number of times to achieve a good binding. The protrusions prevent the particles from interacting with any other binding site, thereby, as already mentioned above, reducing or eliminating cross-reactivity problems.

Preferred lateral movement sequences are typically defined such that the interaction of the particles with the binding sites is as high as possible. This means that one would typically move the particles in another direction as soon as a significant number of them has run into the barrier. Besides changing the direction of the lateral field gradient, the magnetic field is typically also switched on and off as a function of time, resulting in a pulsed actuation sequence.

Besides cross-reactivity problems, the protrusions can also prevent uniformity problems concerning the distribution of particles on the detection surface caused by, for example, an inhomogeneous magnetic field generated by the magnetic unit. The design of a coil for magnetic actuation is a trade-off between force and uniformity. For point-of-care testing the coil would typically be optimised for speed. As a result the magnetic field gradient is not uniform over the detection surface. This could lead to a concentration of the particles near the middle of the detection surface. As a consequence, the effective detection surface area could be reduced to a smaller area, in which the particles are concentrated, since only in this location the particles have significant surface interaction. The protrusions can prevent the particles from collecting all at certain locations on the detection surface, in particular in the middle of the detection surface, thereby increasing the effective area of the detection surface.

Although in the above described embodiment the particle detection unit for detecting the particles on the detection surface is an optical particle detection unit using the FTIR technique, in other embodiments other particle detection units can be used. The particle detection unit can be any suitable particle detection unit to detect the presence of the particles, in particular of magnetic particles, on or near to the detection surface, based on any property of the particles. For example, the particle detection unit can use i) magnetic methods based on, for example, magneto-resistive properties or/and the Hall-effect, optical methods based on imaging, fluorescence, chemiluminescence, absorption, scattering, evanescent field techniques, surface plasmon resonance, Raman effect etc., ii) sonic detection methods based on, for example, a surface acoustic wave, a bulk acoustic wave, cantilevers, a quartz crystal etc., iii) electric detection methods based on, for example, conduction, impedance, amperometric, redox cycling etc. and iv) combinations of these methods. If magnetic methods are used by the particle detection unit for detecting magnetic particles on the detection surface, the particle detection unit comprises preferentially a coil, a magneto-resistive sensor, a magneto-restrictive sensor, a Hall sensor, in particular, a planar Hall sensor, a flux gate sensor, a SQUID and/or a magnetic resonance sensor etc.

The embodiments described in the present invention can be used as rapid, robust and easy to use point-of-care biosensors, in particular, for small sample volumes. The detection cavity, which can be regarded as a reaction chamber, can be a disposable item to be used together with a compact reader containing the magnetic unit and the particle detection unit. Furthermore, the apparatuses and methods of the present invention can be used in automated high-throughput testing. In this case, the detection cavity is, for example, a well plate or a cuvette, fitting into an automated instrument.

The particles are preferentially magnetic particles. It is further preferred that the particles are nano particles, which have preferentially at least one dimension ranging between 3 nm and 5000 nm, further preferred between 10 nm and 3000 nm and further preferred between 50 nm and 1000 nm.

Although in the above described embodiments a certain number of areas surrounded by the protrusions are schematically and exemplarily shown in the Figures, the invention is not limited to a certain number of areas and protrusions. Furthermore, although in the above described embodiments one or two kinds of binding sites and particle providing units are present, in other embodiments more different kinds of binding sites and more different kinds of particle providing units can be present, in order to detect different kinds of target elements by detecting the particles on and, in particular, near to the detection surface. In a preferred embodiment, different kinds of binding sites and particle providing units are present in the same amount, i.e. each kind of binding site and particle providing unit is present in the same amount.

Other variations to the disclosed embodiments can be understood and effected by those skilled in the art and practising the claimed invention, from the study of the drawings, the disclosure and the appended claims.

In the claims, the word “comprising” does not exclude other elements or steps, and the indefinite article “a” or “an” does not exclude a plurality.

A single unit or device may fulfil the functions of several items recited in the claims. The mere facts that certain measures are recited in mutually different dependent claims, does not indicate that a combination of these measures can not be used to advantage.

A computer program may be stored/distributed on a suitable medium, such as an optical storage medium or a solid-state medium, supplied together with or as part of other hardware, but may also be distributed in other forms, such as via the internet or other wired or wireless telecommunication systems.

Any reference signs in the claims should not be construed as limiting the scope.

1. A detection apparatus (1) for detecting particles (25), wherein the detection apparatus (1) comprises a detection surface (30) for detecting particles on the detection surface and wherein the detection surface (30) comprises protrusions (40) for limiting a movement of the particles (25).

2. The detection apparatus as claimed in claim 1, wherein the protrusions (40) divide the detection surface into areas (41) surrounded by the protrusions such that a particle of the particles (25) located on the detection surface (30) is maintained within the respective area, if the particle moves laterally on the detection surface (30).

3. The detection apparatus as claimed in claim 1, wherein the detection surface comprises binding sites (42, 43) for binding the particles (25).

4. The detection apparatus as claimed in claim 3, wherein the detection surface (30) comprises different kinds (42, 43) of binding sites comprising different binding properties.

5. The detection apparatus as claimed in claim 1, wherein the detection apparatus (1) comprises a magnetic unit (19), wherein the particles (25) are magnetic particles and wherein the magnetic unit (19) is adapted for generating a magnetic field for forcing the particles (25) onto the detection surface (30).

6. The detection apparatus claimed in claim 3, wherein the detection apparatus comprises a magnetic unit (19) for generating a magnetic field for forcing the particles (25), which are not bound to the detection surface (30), away from the detection surface (30).

7. The detection apparatus as claimed in claim 1, wherein the detection apparatus (1) comprises a particle providing unit (44, 45) for providing the particles (25), wherein the particle providing unit (44, 45) is adapted for providing particles (25), which comprise an attaching element (27, 127) for attaching the particle (25) to a target element (28, 128), wherein the detection surface (30) comprises binding sites (42, 43) for binding the particles (25) and wherein the binding sites (42, 43) are adapted such that a particle of the particles (25) is bindable to a binding site (42, 43), if the particle (25) is attached to the target element (28, 128).

8. The detection apparatus as claimed in claim 7, wherein the detection apparatus (1) comprises a detection cavity (14), in which a fluid (3) comprising target elements (28, 128) and the particles (25) provided by the particle providing unit (44, 45) are introducable for allowing the attaching elements (27, 127) of the particles (25) to attach to the target elements (28, 128) of the fluid (3), wherein the detection apparatus (1) is adapted for exposing the fluid (3) comprising the target elements (28, 128) attached to the particles (25) to the detection surface (30) comprising the binding sites (42, 43).

9. The detection apparatus as claimed in claim 7, wherein the protrusions (40) define areas (41), wherein in at least one area a binding site (42) is located, which differs from a binding site (43) located in another at least one area with respect to binding properties, wherein the detection apparatus (1) comprises different particle providing units (44, 45) for providing particles with different kinds (27, 127) of attaching elements, wherein the different kinds (27, 127) of attaching elements correspond to the different kinds of binding sites (42, 43) such that a particle with an attaching element binds mainly to the corresponding binding site.

10. The detection apparatus as claimed in claim 9, wherein in a same area of the areas (41) on the detection surface (30) a particle providing unit (44) is located, which provides particles with a kind of attaching elements (127), which corresponds to a binding site (43) in the same area.

11. The detection apparatus as claimed in claim 9, wherein the detection apparatus (1) comprises a particle providing surface (150), on which the particle providing units (144, 145) are located, wherein the particle providing surface (150) is located opposite to the detection surface (130) and wherein the particle providing units (144, 145) are arranged opposite to the corresponding binding sites (142, 143).

12. The detection apparatus as claimed in claim 1, wherein the detection apparatus comprises a particle detection unit (20 . . . 23) for detecting the particles (25) on the detection surface (30; 130).

13. A detection method for detecting particles (25), wherein the detection method comprises the steps of detecting particles (25) on a detection surface (30) comprising protrusions (40) for limiting a movement of the particles (25).

14. A method for producing a detection apparatus for detecting particles (25), the method comprising following steps:
providing a detection surface material for producing a detection surface (30) for detecting particles (25) on the detection surface (30),
forming a detection surface from the provided detection surface material such that the detection surface (30) comprises protrusions (40) for limiting a movement of the particles (25).

15. A computer program for detecting particles (25); the computer program comprising program code means for causing a detection apparatus as defined in claim 1 to carry out the steps of the detection method for detecting particles (25), wherein the detection method comprises the steps of detecting particles (25) on a detection surface (30) comprising protrusions (40) for limiting a movement of the particles (25), when the computer program is run on a computer controlling the detection apparatus.
